(19) United States
(12) Patent Application Publication
(10) Pub. No.: US 2018/0242063 A1
(43) Pub. Date: Aug. 23, 2018

(54) WIRELESS BONE CONDUCTION RING FOR VOICE TRANSMISSION AND RECEIVING

(71) Applicants: Yueh-Heng Chiang, Hsinchu City (TW); Yi-Teng Pu, New Taipei (TW)

(72) Inventors: Yueh-Heng Chiang, Hsinchu City (TW); Yi-Teng Pu, New Taipei (TW)

(21) Appl. No.: 15/435,307

(22) Filed: Feb. 17, 2017

(51) Int. Cl. H04R 1/02; H04R 17/02; H04R 1/04; H04R 1/28; H04R 19/00; H04R 3/02; H02J 7/02; A61B 5/00; A61B 5/11

(52) U.S. Cl. CPC H04R 1/028; H04R 17/02; H04R 1/04; H04R 1/2876; H04R 19/00; H04R 3/02; A61B 2562/0219; A61B 5/0059; A61B 5/11; A61B 5/0015; H04R 2460/13; H04R 2201/023; H04R 2420/07; H02J 7/025

(57) ABSTRACT

A wireless bone conduction ring for voice transmission and receiving includes a body having an annular structure and capable of being worn on a finger; a wireless receiver installed on the body for communication with other electronic device; a bone conduction voice to vibration converter installed on the body and connected to the wireless receiver; the bone conduction voice to vibration converter receiving voice signals from the wireless receiver and then generating mechanical vibrations according to the received voice signals; wherein when a user's finger contacts skins near his or her ear, the vibration will be transferred to the head bone of the user through the finger wearing the ring; then the vibration is further transferred to the user's hearing organisms so as to have hearing sense; and a battery installed in the body and connected to the wireless receiver for power supply.

### FIELD OF THE INVENTION

The present invention is related to bone conduction devices, and in particular to a wireless bone conduction ring for voice transmission and receiving.

### BACKGROUND OF THE INVENTION

Bone conduction is a technology for transferring voices signals into mechanic vibrations of different frequencies. Then the vibrations are transferred to the hearing sensor organs in the brain to understand the voices. As comparing the conventional voice transformation way, the bone transferring of the voice does not use air wave as a medium. Therefore environmental noises do not interfere the voices. Furthermore, the voices transferred by bones do not disperse due to transfer in air. Thus, currently, bone transfer for voices has been used in trumpets, such as bone conduction trumpet.

A bone voice transfer trumpet converts electric energy into vibrations. Then the vibration element contacts skins of user's head so that the vibration is transferred to the ear diaphragm. Then the vibration is further transferred and is converted into hearing sense signals. Currently, this technology is used in bone conduction earphone. In this kind earphone, a bone transfer trumpet is used to replace the conventional coil trumpets. In use, the bone conduction trumpet must be tightly adhered to the skin of user's head for getting preferred effect.

A critical defect of a bone conduction earphone is that the user cannot suffer from the uneasy feeling as the bone conduction trumpet adhered to the skin for a long time. Currently some smart watch is arranged with bone conduction trumpet. In using the trumpet, a finger of the hand wearing the watch is adhered to the head skin. Vibrations from the watch are transferred from the forearm, wrist, palm, and the finger to the head bone. The path for vibration transferring is too long to have a preferred effect. Furthermore, the watch is tightly adhered to a user's wrist so that the user will feel uneasy.

### SUMMARY OF THE INVENTION

Accordingly, the object of the present invention is to improve the defects aforesaid, and therefore the present invention provides a wireless bone conduction ring for voice transmission and receiving, wherein in the present invention, the bone conduction voice to vibration converter is worn on the user's finger so that the user will not feel uneasy. Only in receiving, the finger contacts the skin near the ear. Total time period is short and the user will not feel uneasy. Furthermore, with respect to the bone conduction trumpet worn on a watch, the present invention causes the bone conduction voice to vibration converter is worn on a finger. In use, the finger contacts the skin near the ear. The overall transfer path is short and decay of the vibration is finite. Therefore the quality of the voice is clear. The defect in the prior art is improved greatly.

To achieve above object, the present invention provides a wireless bone conduction ring for voice transmission and receiving. The wireless bone conduction ring for voice transmission and receiving of the present invention includes a body having an annular structure and capable of being worn on a finger; a wireless receiver installed on the body for communication with other electronic device; a bone conduction voice to vibration converter installed on the body and connected to the wireless receiver; the bone conduction voice to vibration converter receiving voice signals from the wireless receiver and then generating mechanical vibrations according to the received voice signals; wherein when a user's finger contacts skins near his or her ear, the vibration will be transferred to the head bone of the user through the finger wearing the ring; then the vibration is further transferred to the user's hearing organisms so as to have hearing sense; and a battery installed in the body and connected to the wireless receiver for power supply.

### BRIEF DESCRIPTION OF THE DRAWINGS

FIG. 1 shows the embodiment of the present invention.

FIG. 2 is a structural schematic view of FIG. 1 of the present invention.

FIG. 3 is a structural block diagram of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

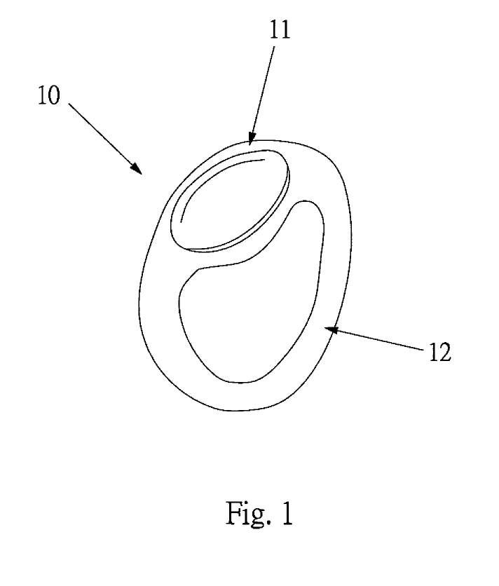

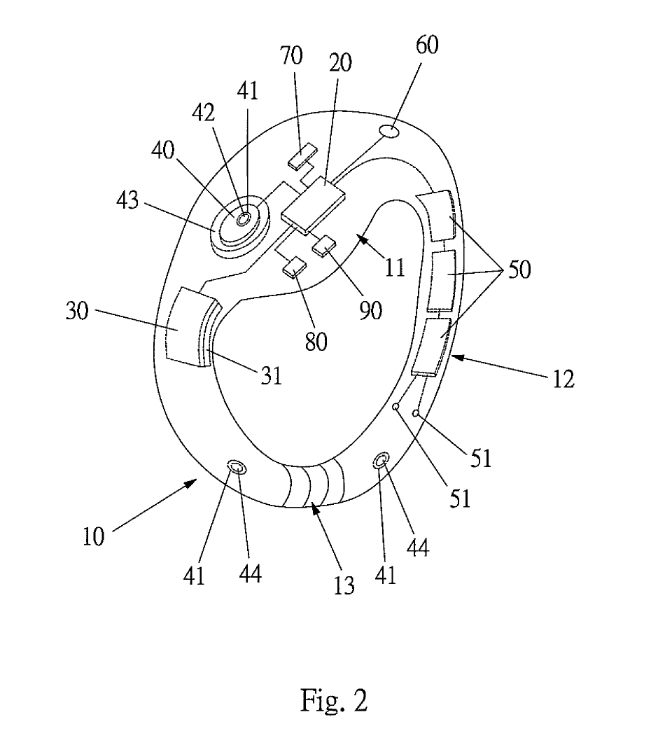

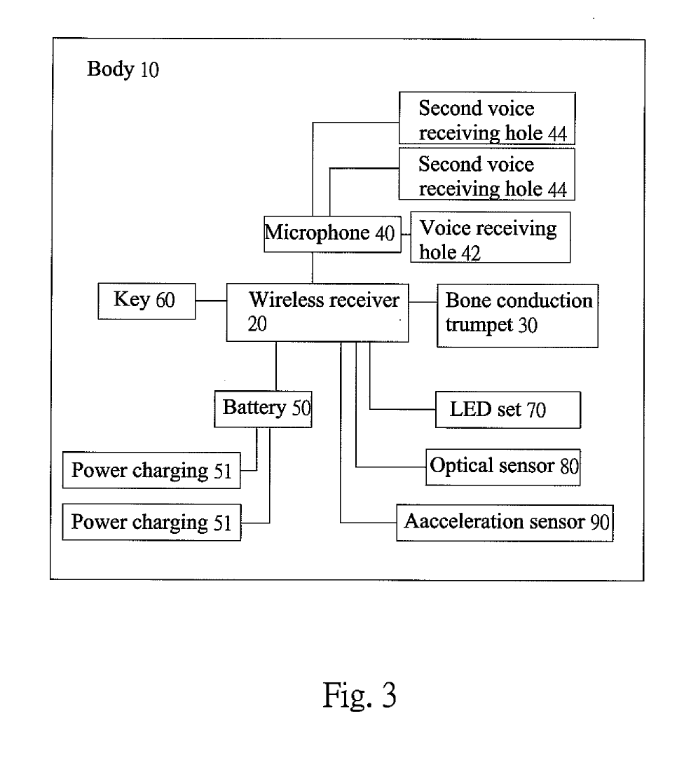

With reference to FIGS. 1 to 3, the ring with bone conduction receiver of the present invention is illustrated. The present invention includes the following elements.

A body 10 has an annular structure and capable of being worn on a finger. Preferably, the body 10 is a ring with a beautiful outlook and having a decoration thereon, such as a jewel is added thereon.

The body 10 has an enlarged portion 11 and a smaller annular section 12.

A wireless receiver 20 is installed on the body 20 for communication with other electronic device, such as a handset. The wireless receiver 20 is generally a blue tooth device.

A bone conduction voice to vibration converter 30 is on the body 20 and is connected to the wireless receiver 20. The bone conduction voice to vibration converter 30 receives voice signals from the wireless receiver 20 and then generates mechanical vibrations according to the received voice signals. When a user's finger contacts skins near the ear, vibration will be transferred to the head bone of the user through the finger wearing the ring. Then the vibration is further transferred to the user's hearing organisms so as to have hearing sense. Therefore the user receives the voice.

The present invention further includes a vibration soft medium 31 which is attached to a surface of the bone conduction voice to vibration converter 30 for contacting user's finger. The vibration from the bone conduction voice to vibration converter 30 is transferred to the finger wearing the body 10 through the vibration soft medium 31. Preferably, the vibration soft medium 31 is made of TPE.

A microphone 40 is in the body 10 and is connected to the wireless receiver 20 for receiving user's voices and transferring the voice outwards through the wireless receiver 20. The microphone 40 has a voice receiving hole 42 for receiving voices. The voice receiving hole 42 is installed with a breathable impermeable membrane 41. Furthermore, a position of the voice receiving hole 42 of the microphone 40 is preferably not to be shielded a by user's finger.

The microphone 40 may be a piezoelectric microphone which captures vice signals with a single one vibrating diaphragm so that no water proof membrane is used.

Furthermore, the wireless receiver 20 has a function of feedback suppression for reducing the echo when the microphone 40 captures the voice signals from the bone conduction voice to vibration converter 30.

The present invention further includes a soft rubber cover 43 for enclosing the microphone 40 and absorbing vibrations from the bone conduction voice to vibration converter 30. Preferably, the soft rubber cover 43 encloses the microphone 40 and do not shield the voice receiving hole 42. Preferably, the soft rubber cover 43 is made of TPE.

In the present invention, the body 10 is formed with a second voice receiving hole 44 which is connected to the microphone 40 and is used to reduce the environmental noises. The second voice receiving hole 44 serves to receive echo or noises from different aspects and feedback the echo and noises received to the wireless receiver 20 for calibrating the voice received by the microphone 40 so as to reduce the level of the noises. In the present invention, the second voice receiving hole 44 is equipped with a breathable impermeable membrane 41.

A battery 50 is installed on the body 10 and is connected to the wireless receiver 20 for power supply. Preferably, the battery is a lithium battery. Preferably, the battery 50 is distributed on the annular section 12 and preferably the battery 50 is divided into several sections so as to reduce the whole size, increase the whole power capacity and is equipped in the annular section 12.

The battery 50 is chargeable and can be charged wirelessly. Furthermore, a periphery of the body 10 is installed with at least one port 51 for power charging externally. For example, there are two ports 51 for power charging.

Other than above mentioned elements, the present invention further includes the following elements.

A key 60 is as a switch for actuating the body 10, wireless connection (for example, blue tooth connection, and receiving phones.

An LED set 70 serves to display the operation states of the body 10, for example, blue tooth connection between the body 10 and a wireless communication device.

A telescopic unit 13 is installed on the body 10 for adjusting the tightness of the body 10.

An optical sensor 80 is installed on the body 10 and is connected to the wireless receiver 20 for detecting physical condition of a human body.

An acceleration sensor 90 (such as a G sensor) is positioned on the body 10 for detecting the pose of user's hand so as to actuate or stop a communication.

In use of the present invention, the user uses his (or her) finger wearing the ring body 10 of the present invention to touch a peripheral skin near the ear so as to transfer the vibration of the bone conduction voice to vibration converter 30 to the head bone and then to the ear membrane so that the user can hear voices from the wireless receiver 20. Or the finger of the user can put into the hole of ear so as to reduce the noise from the environment and increase the resolution of the voice received.

Advantages of the present invention are that in the present invention, the bone conduction voice to vibration converter is worn on the user's finger so that the user will not feel uneasy. Only in receiving, the finger contacts the skin near the ear. Total time period is short and the user will not feel uneasy. Furthermore, with respect to the bone conduction voice to vibration converter worn on a watch, the present invention causes the bone conduction voice to vibration converter is worn on a finger. In use, the finger contacts the skin near the ear. The overall transfer path is short and decay of the vibration is finite. Therefore the quality of the voice is clear. The defect in the prior art is improved greatly.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A wireless bone conduction ring for voice transmission and receiving, comprising:
a body having an annular structure and capable of being worn on a finger;
a wireless receiver installed on the body for communication with other electronic device;
a bone conduction voice to vibration converter installed on the body and connected to the wireless receiver; the bone conduction voice to vibration converter receiving voice signals from the wireless receiver and then generating mechanical vibrations according to the received voice signals; wherein when a user's finger contacts skins near his or her ear, the vibration will be transferred to the head bone of the user through the finger wearing the ring; then the vibration is further transferred to the user's hearing organisms so as to have hearing sense; and
a battery installed in the body and connected to the wireless receiver for power supply.

2. wireless bone conduction ring for voice transmission and receiving as claimed in claim 1, further comprising a microphone installed in the body and connected to the wireless receiver for receiving user's voices and transferring the voice outwards through the wireless receiver and the wireless receiver is a blue tooth device.

3. The wireless bone conduction ring for voice transmission and receiving as claimed in claim 1, wherein the battery is divided into several sections and is distributed in the annular structure of the body.

4. The wireless bone conduction ring for voice transmission and receiving as claimed in claim 1, further comprising a vibration soft medium attached to a surface of the bone conduction voice to vibration converter; vibrations from the bone conduction voice to vibration converter is transferred to the finger wearing the body through the vibration soft medium.

5. The wireless bone conduction ring for voice transmission and receiving as claimed in claim 2, wherein the microphone has a voice receiving hole for receiving voices; the voice receiving hole is installed with a breathable impermeable membrane; and a position of the voice receiving hole of the microphone is not to be shielded a by user's finger.

6. The wireless bone conduction ring for voice transmission and receiving as claimed in claim 2, further comprising a soft rubber cover for enclosing the microphone and absorbing vibrations from the bone conduction voice to vibration converter; and the soft rubber cover enclosing the microphone and do not shield the voice receiving hole.

7. The wireless bone conduction ring for voice transmission and receiving as claimed in claim 1, wherein the battery is charged wirelessly.

8. The wireless bone conduction ring for voice transmission and receiving as claimed in claim 1, wherein a periphery of the body is equipped with at least one point charge port for charging electrically form external power source.

9. The wireless bone conduction ring for voice transmission and receiving as claimed in claim 1, further comprising:
a key as a switch for actuating the body, wireless connection, and receiving phones.

10. The wireless bone conduction ring for voice transmission and receiving as claimed in claim 1, further comprising:
an LED set for displaying operation states of the body.

11. The wireless bone conduction ring for voice transmission and receiving as claimed in claim 1, further comprising:
a telescopic unit installed on the body for adjusting the tightness of the body.

12. The wireless bone conduction ring for voice transmission and receiving, further comprising:
an optical sensor installed on the body and connected to the wireless receiver for detecting physical condition of a human body.

13. The wireless bone conduction ring for voice transmission and receiving as claimed in claim 1, wherein the wireless receiver is a blue tooth device.

14. The wireless bone conduction ring for voice transmission and receiving as claimed in claim 1, wherein the body is a ring and has a decoration thereon.

15. The wireless bone conduction ring for voice transmission and receiving as claimed in claim 1, wherein the microphone is a piezoelectric microphone which captures vice signals with a single one vibrating diaphragm without using any water proof membrane.

16. The wireless bone conduction ring for voice transmission and receiving as claimed in claim 1, wherein the body is formed with a voice receiving hole which is connected to the microphone and is used to reduce the environmental noises; the second voice receiving hole serves to receive echo or noises from different aspects and feedback the echo and noises received to the wireless receiver for calibrating the voice received by the microphone so as to reduce the level of the noises.

17. The wireless bone conduction ring for voice transmission and receiving as claimed in claim 1, wherein the wireless receiver has a function of feedback suppression for reducing the echo when the microphone captures the voice signals from the bone conduction voice to vibration converter.
